(19) United States
(12) Patent Application Publication
(10) Pub. No.: US 2025/0281694 A1
(43) Pub. Date: Sep. 11, 2025

(54) SYSTEM AND METHOD FOR SWITCHING BETWEEN CLOSED LOOP AND OPEN LOOP CONTROL OF AN AMBULATORY INFUSION PUMP

(71) Applicant: Tandem Diabetes Care, Inc., San Diego, CA (US)

(72) Inventor: Michael J. Rosinko, Anaheim, CA (US)

(21) Appl. No.: 19/215,916

(22) Filed: May 22, 2025

Related U.S. Application Data
(63) Continuation of application No. 18/128,830, filed on Mar. 30, 2023, now Pat. No. 12,318,578.
Application No. 18/128,830 is a continuation of application No. 16/791,129, filed on Feb. 14, 2020, now Pat. No. 11,638,781.
Application No. 16/791,129 is a continuation of application No. 15/394,066, filed on Dec. 29, 2016, now Pat. No. 10,569,016.
(60) Provisional application No. 62/272,255, filed on Dec. 29, 2015.

(51) Int. Cl. A61M 5/172; A61M 5/142

(52) U.S. Cl. CPC A61M 5/1723; A61M 5/14244; A61M 2205/3553; A61M 2205/3569; A61M 2205/3584; A61M 2205/3592; A61M 2205/505; A61M 2205/52; A61M 2205/702; A61M 2230/005; A61M 2230/201

(57) ABSTRACT

An infusion pump system providing therapy to a patient in a closed-loop or semi-closed loop mode can safely automatically revert to open-loop therapy. The system stores a default open- loop basal rate profile in memory. The system also continually tracks the insulin on board for the patient over a plurality of closed-loop therapy intervals. When an error or event occurs requiring reversion to open-loop therapy, the system automatically provides therapy according to the open- loop basal rate profile and the tracked insulin on board amount.

### RELATED APPLICATIONS

This application is a continuation of application Ser. No. 18/128,830, filed Mar. 30, 2023, which in turn is a continuation of application Ser. No. 16/791,129, filed Feb. 14, 2020, which is now U.S. Pat. No. 11,638,781, issued May 2, 2023, which in turn is a continuation of application Ser. No. 15/394,066, filed Dec. 29, 2016, which is now U.S. Pat. No. 10,569,016, issued Feb. 25, 2020, which claims the benefit of U.S. Provisional Application No. 62/272,255 filed Dec. 29, 2015, which is hereby incorporated herein in its entirety by reference.

### FIELD OF THE INVENTION

The present invention relates generally to ambulatory infusion pumps and, more particularly, to ambulatory infusion pumps that are able to safely transition from closed loop operation to open-loop operation.

### BACKGROUND OF THE INVENTION

There are many applications in academic, industrial, and medical fields that benefit from devices and methods that are capable of accurately and controllably delivering fluids, such as liquids and gases that have a beneficial effect when administered in known and controlled quantities. Such devices and methods can be particularly useful in the medical field where treatments for many patients include the administration of a known amount of a substance at predetermined intervals.

One category of devices for delivering such fluids is that of pumps that have been developed for the administration of insulin and other medicaments for those suffering from both type I and type II diabetes. Some pumps configured as portable infusion devices can provide continuous subcutaneous medicament injection and/or infusion therapy for the treatment of diabetes. Such therapy may include, e.g., the regular and/or continuous injection or infusion of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Portable infusion pumps for delivering insulin or other medicaments can be used in conjunction with blood glucose monitoring systems, such as blood glucose meters (BGMs) and continuous glucose monitoring devices (CGMs). A CGM provides a substantially continuous estimated blood glucose level through a transcutaneous sensor that estimates blood analyte levels, such as blood glucose levels, via interrogation of the patient's interstitial fluid rather than the patient's blood. CGM systems typically consist of a transcutaneously-placed sensor, a transmitter and a monitor. A CGM system allows a patient or caregiver to insert a single sensor probe under the skin for multiple days. Thus, the patient is only required to perform a single moderately invasive action with a single entry point in the subdermal layer on, e.g., a weekly basis.

Ambulatory infusion pumps typically allow the patient or caregiver to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, automatically temporarily ceasing or prompting the user temporarily to cease insulin administration. These portable pumps may incorporate a BGM or CGM within the hardware of the pump or may communicate with a dedicated BGM or CGM via wired or wireless data communication protocols, directly and/or via a device such as a smartphone. Such pumps may be particularly important in facilitating patient compliance and improved or more accurate treatment of diabetes. One example of integration of infusion pumps with CGM devices is described in U.S. Patent Publication No. 2014/0276419, which is hereby incorporated by reference herein.

The delivery of insulin or other medicament from a portable infusion pump making use of CGM data necessitates accurate and reliable CGM data output. Some CGM devices are calibrated with blood samples to correlate actual blood glucose data with the CGM readings. However, such calibrations are only done periodically, such as every few days or hours, such as 12 hours, and the longer it has been since a calibration event the more likely the CGM is unreliable to some degree and the more unreliable the CGM is likely to become until the next calibration.

As noted above, insulin or other medicament dosing by basal rate and/or bolus techniques could automatically be provided by a pump based on readings received into the pump from a CGM device that is, e.g., external to the portable insulin pump or integrated with the pump as a pump- CGM system in a closed-loop or semi-closed-loop fashion. With respect to insulin delivery, some systems including this feature can be referred to as artificial pancreas systems because the systems serve to mimic biological functions of the pancreas for patients with diabetes.

However, there are a number of risks in automatically dosing insulin, or other medicaments, based on CGM readings that may be inaccurate or unreliable. For example, a CGM reading or readings may indicate that a user's blood glucose level is high and therefore the pump may automatically deliver a bolus of a medicament such as insulin or increase the basal rate of a medicament such as insulin to lower the user's blood glucose to a target level. If the CGM reading inaccurately indicates that the user's blood glucose level is high, the extra insulin delivered in response may actually lower the user's blood glucose level below a desired target level, possibly to a dangerously low level. This problem may not be detected until the CGM is next calibrated, perhaps not for several hours. Thus, automatically dosing medicaments such as insulin based on CGM readings can have potentially dangerous effects in situations where the CGM readings are inaccurate or unreliable relative to the user's actual blood glucose levels. Similarly, any failure of the CGM sensor, loss of signal or communication between the CGM and the pump, other mechanical or electrical failures with the system or problems with the user's operation of the system or its components, for example, may also be dangerous to the patient.

Thus, a need exists for devices and methods that maintain safe delivery of insulin to a patient in the event of a failure of closed-loop or semi-closed-loop automatic dosing of an insulin pump.

### SUMMARY OF THE INVENTION

An infusion pump system providing therapy to a patient in a closed-loop or semi-closed loop mode can safely automatically revert to open-loop therapy. The system stores a default open- loop basal rate profile in memory. The system also continually tracks the insulin on board for the patient over a plurality of closed-loop therapy intervals. When an error or event occurs requiring reversion to open-loop therapy, the system automatically provides therapy according to the open- loop basal rate profile and the tracked insulin on board amount.

In one embodiment, an infusion pump system includes a pump mechanism, a memory that stores a default open-loop basal rate profile and a communications device configured to receive information from a continuous glucose monitoring system (CGM). A processor of the system causes the pump mechanism to deliver medicament to the patient in a closed-loop manner based on the information from the CGM, while continually tracking an amount of insulin on board in the patient. When an error requiring reversion from closed-loop mode to open-loop mode is detected, the processor causes the pump mechanism to deliver medicament according to the open-loop basal rate profile stored in memory and the amount of insulin on board in the patient tracked during the closed-loop mode.

In one embodiment, an infusion pump system includes a pump mechanism and a memory that stores a default open-loop basal rate profile. A processor of the system receives information from a CGM and causes the pump mechanism to automatically deliver medicament to the patient based on therapy parameters automatically determined based on the information from the CGM while continually tracking an amount of insulin on board in the patient. When an error pertaining to the CGM occurs, the pump mechanism ceases automatically delivering medicament based on the determined therapy parameters and delivers medicament according to the open-loop basal rate profile stored in memory and the tracked amount of insulin on board in the patient.

In some embodiments, the default open-loop basal rate profile is continually updated. The closed-loop therapy can be provided over a plurality of time intervals. The insulin on board can be tracked for each time interval. The open-loop basal rate profile can be updated after each time interval based on the respective tracked insulin on board amount.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention may be more completely understood in consideration of the following detailed description of various embodiments of the invention in connection with the accompanying drawings, in which:

FIG. 1 is a medical device that can be used with embodiments of the present invention.

FIG. 2 is a block diagram representing a medical device that can be used with embodiments of the present invention.

FIG. 3 depicts an exemplary screen shot of a home screen page of a user interface for use with an infusion pump system that can be used with embodiments of the present invention.

FIG. 4 is a schematic representation of a system according to embodiments of the present invention.

FIG. 5 is a flowchart of a method of operating a pump in a closed-loop mode according to an embodiment of the present invention.

FIG. 6 is a flowchart of a method of reverting to open-loop mode according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

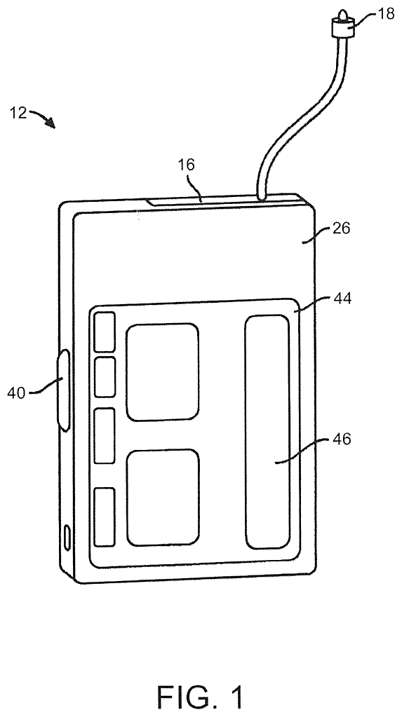

FIG. 1 depicts an embodiment of a medical device that can be used with embodiments of the present invention. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. When output/display 44 is of the visual type, it may comprise an LCD display, LED display, plasma display, graphene-based display, OLED display or the like. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote control device, a laptop computer, personal computer, tablet computer, a mobile communication device such as a smartphone, a wearable electronic watch or electronic health or fitness monitor, or personal digital assistant (PDA), a CGM display etc.

Such one or more other display devices may be configured to be used in place of output/display 44 or to work in connection with output/display 44 such that information may be repeated in exact or similar fashion between output/display 44 and one or more other displays, such that different information may be repeated between/among output/display 44 and one or more other display devices, or such that information is presented solely on one or more other display devices. Such one or more other display devices may also include the capability to allow a user to input information and/or commands for operation of the infusion pump, such as, e.g., via a touchscreen, microphone, keyboard or other input devices as are known in the art.

In one embodiment, the medical device can be a portable insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient. In a further embodiment, the medical device can be a glucose meter such as a BGM or CGM. Further detail regarding such systems and definitions of related terms can be found in, e.g., U.S. Pat. Nos. 8,311,749, 7,711,402 and 7,497,827, each of which is hereby incorporated by reference herein in its entirety. In other embodiments, the medical device can monitor other physiological parameters of a patient.

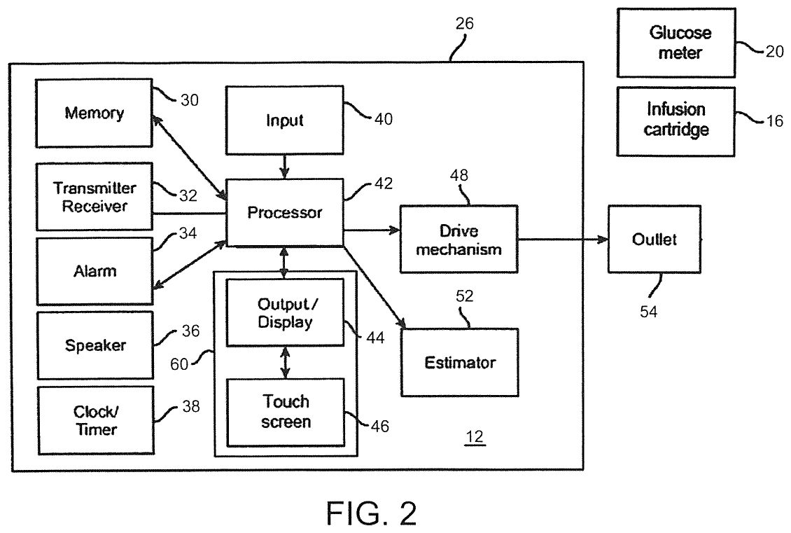

FIG. 2 illustrates a block diagram of some of the features that can be used with embodiments of the present invention, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from an input device, such as a sensor that may sense pressure, temperature or other parameters.

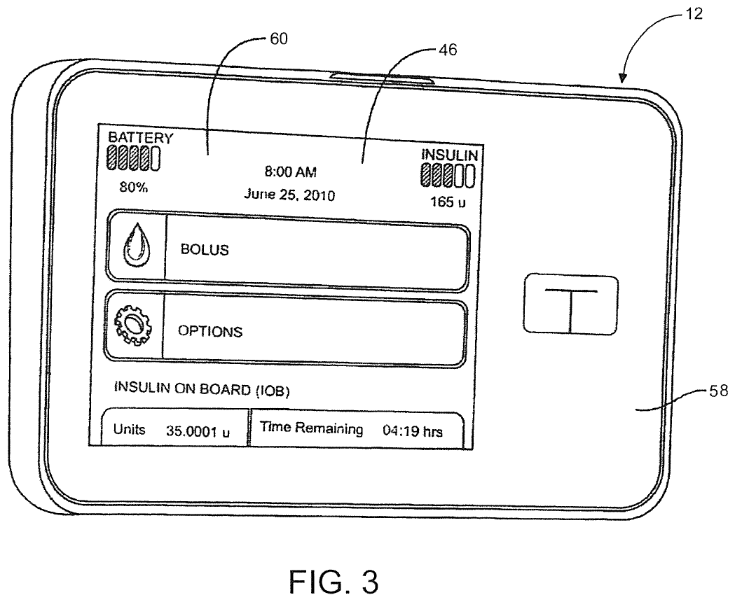

Referring to FIG. 3, a front view of pump 12 is depicted. Pump 12 may include a user interface, such as, for example, a GUI 60 on a front surface 58 or other location of pump 12. GUI 60 may include a touch-sensitive screen 46 that may be configured for displaying data, facilitating data and/or command entry, providing visual tutorials, as well as other interface features that may be useful to a caregiver or to the patient operating pump 12. The GUI can also present alarms or alerts to the user. Although described with respect to infusion pump 12, such a GUI 60 could additionally or alternatively be employed on any other device employed as part of an infusion pump system such as, for example, a CGM (described below), dedicated remote controller, smartphone, electronic tablet, computer, etc.

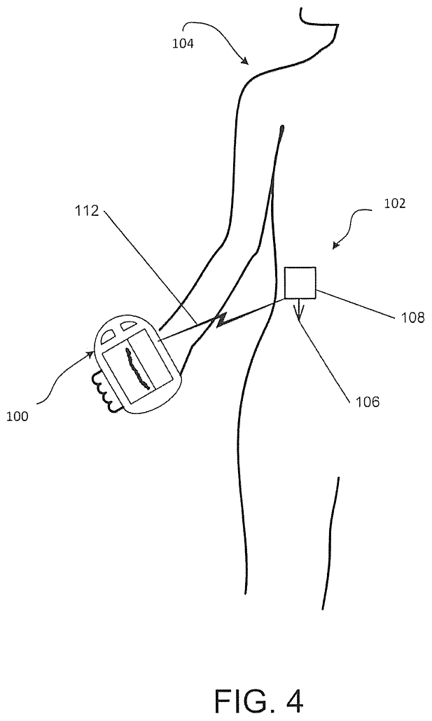

Pump 12 can interface directly or indirectly (via, e.g., a smartphone or other device) with a glucose meter, such as a blood glucose meter (BGM) or a continuous glucose monitor (CGM); the latter category of which provides a substantially continuous estimated blood glucose level through a transcutaneous sensor that estimates blood analyte levels, such as blood glucose levels, via interrogation of the patient's interstitial fluid rather than the patient's blood. Referring to FIG. 4, an exemplary CGM system 100 according to an embodiment of the present invention is shown (other CGM systems can be used). The illustrated CGM system includes a sensor 102 affixed to a patient 104 and can be associated with the insulin infusion device 12 in a CGM-pump system. The sensor 102 includes a sensor probe 106 configured to be inserted to a point below the dermal layer (skin) of the patient 104. The sensor probe 106 is therefore exposed to the patient's interstitial fluid or plasma beneath the skin and reacts with that interstitial fluid to produce a signal that can be associated with the patient's blood glucose (BG) level. The sensor 102 includes a sensor body 108 that transmits data associated with the interstitial fluid to which the sensor probe 106 is exposed. The data may be transmitted from the sensor 102 to the glucose monitoring system receiver 100 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 102 to the monitoring system 100. Transmission of sensor data to the glucose monitoring system receiver by wireless or wired connection is represented in FIG. 4 by the arrow line 112. Further detail regarding such systems and definitions of related terms can be found in, e.g., U.S. Pat. Nos. 8,311,749, 7,711,402 and 7,497,827, each of which is hereby incorporated by reference in its entirety.

In one embodiment of a pump-CGM system, part of the CGM system 100 is incorporated into the housing of the pump 12 such that the processor 42 of the pump 12 is adapted to receive the data directly from the sensor 102 through a wired or wireless link and process and display the data on the pump display 44. In another embodiment, the CGM 100 is a separate device that communicates with the pump 12 processor 42 through a wired or wireless link to transmit processed CGM data to the pump 12 for display on the pump display 44. In further embodiments, the CGM system can transmit data to an intermediary device, such as, for example, a smartphone or dedicated remote controller that can then communicate the data to the pump.

In an embodiment of a pump-CGM system having a pump 12 that communicates with a CGM and that integrates CGM data and pump data as described herein, the CGM can automatically transmit the glucose data to the pump. The pump can then automatically determine therapy parameters and deliver medicament based on the data. For example, if the CGM data indicates that the user's blood glucose level is over a high blood glucose threshold level stored in memory, the pump can automatically calculate and deliver an insulin bolus amount and/or an increase to a user's basal rate to bring the user's blood glucose level below the threshold and/or to a target value. As with other parameters related to therapy, such thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

In one embodiment, such an automatic pump-CGM system for insulin delivery is referred to as an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). For example, if the CGM indicates that the user has a high blood glucose level or hyperglycemia, the system can automatically calculate an insulin dose necessary to reduce the user's blood glucose level below a threshold level or to a target level and automatically deliver the dose. Alternatively, the system can automatically suggest a change in therapy upon receiving the CGM data such as an increased insulin basal rate or delivery of a bolus, but can require the user to accept the suggested change prior to delivery rather than automatically delivering the therapy adjustments.

If the CGM data indicates that the user has a low blood glucose level or hypoglycemia, the system can, for example, automatically reduce a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the user, e.g., ingest carbohydrates and/or take other actions and/or make other suggestions as may be appropriate to address the hypoglycemic condition, singly or in any desired combination or sequence. Such determination can be made by the infusion pump providing therapy or by a separate device that transmits therapy parameters to the infusion pump. In some embodiments, multiple medicaments can be employed in such a system as, for example, a first medicament, e.g., insulin, that lowers blood glucose levels and a second medicament, e.g., glucagon, that raises blood glucose levels.

Because such artificial pancreas systems that incorporate CGM data automatically to adjust insulin therapy in a closed-loop fashion rely on the CGM data to be sufficiently accurate, it may be desirable to implement various features to, e.g., ensure the safety of the patient. Embodiments of the present invention therefore incorporate a temporary suspend feature for artificial pancreas and any other systems that provide closed-loop or semi-closed-loop therapy in which CGM data is relied upon, in whole or in part, automatically to determine dosing information. Semi-closed- loop therapy can include systems that provide some functions on an automatic, closed-loop basis and other functions on a manual or open-loop basis. For example, a system could automatically adjust basal delivery in a closed-loop mode as discussed above while still providing for manual administration of boluses. A system such as that described previously (that automatically suggests a change in therapy upon receiving CGM data such as an increased insulin basal rate or delivery of a bolus, but that requires the user to accept the suggested change prior to delivery rather than automatically delivering the therapy adjustments) could also be considered as providing semi- closed-loop therapy.

During operation of pump 12 in closed-loop mode, it may be desirable to revert to a conventional open-loop delivery mode under certain circumstances. Such circumstances may include, for example, a calibration error with sensor 102, a failure of sensor 102, a loss of signal between sensor 102 and glucose monitoring system 100, a loss of signal between glucose monitoring system 100 and pump 12, a failure of a user to replace a CGM sensor within the recommended expiration period (e.g., a number of days) such that the CGM sensor has lost adequate accuracy, a failure to calibrate a sensor properly, a failure to calibrate a sensor within a time interval, including a manufacturer's recommended time interval, or any other errors which may affect the accuracy of insulin delivery and/or patient safety.

In order for pump 12 safely to revert to open-loop operation mode, a basal rate profile for the patient should be known and the amount of insulin on board (IOB) in the patient at the time of transition between closed-loop and open-loop should be known. Pump 12 therefore includes a default open-loop basal rate profile 202, optionally stored within memory device 30 or optionally stored elsewhere and accessible by pump 12. In one embodiment, default open-loop basal rate profile 202 is programmed into pump 12 prior to patient use. Optionally, the open-loop basal rate profile may be updated during pump operation, such as at the conclusion of an operating interval 230 as described more fully below, to create an updated open-loop basal rate profile 203.

To determine the amount of IOB during closed-loop operation, pump 12, or other device monitoring the therapy, may continually track the amount of insulin delivered 210 over a period of time, such as an interval 230. Interval 230 may comprise a period of time such as hourly, daily, weekly, or other. In one embodiment, an amount of IOB 220 is calculated by comparing the amount of insulin delivered 210 over interval 230 to default open-loop basal rate profile 202. In another embodiment, the amount of IOB 220 is calculated by comparing the amount of insulin delivered 210 over interval 230 to an updated open-loop basal rate profile 203. In either embodiment, IOB 220 represents the difference between the amount of insulin actually delivered to the patient and a default basal rate. In various embodiments, the system can continually track IOB during closed-loop and/or open-loop operation, that is, repeatedly but with breaks/intervals in between where IOB is not tracked, or can continuously track IOB during closed-loop and/or open-loop operation, that is, constant tracking throughout system operation without interruption.

Optionally, the calculation of IOB 220 may take into account an IOB from one or more previous intervals. For example, if at the beginning of an interval 230, the patient already has a positive IOB value, that value will decrease over interval 230 according to known insulin pharmacokinetic models. The calculated decay of insulin already within the patient over interval 230 may be added to the IOB 220 determined as described above.

In embodiments utilizing updated open-loop basal rate profile 203, pump processor or other device may update profile 203 at the conclusion of an interval 230 as needed. For example, if IOB 220 is outside of a predetermined acceptable range, pump 12 may set updated open-loop basal rate profile 203 to correspond to the amount of insulin delivered 210 over the previous interval 230. In another embodiment, pump 12 may compare profile 203, amount of insulin delivered 210 and/or IOB 220 to determine and set a new updated open-loop basal rate profile 203. In some embodiments, when the actual basal insulin delivered 210 is less than the open-loop basal rate profile 203, the IOB 220 calculated during the interval 230 is not used to update the profile 203, or, alternatively, may be used as a negative contribution to reduce the open-loop basal rate profile 203.

During operation of pump 12 in closed-loop mode, upon occurrence of an event that requires reversion to open-loop mode to maintain patient safety, one of basal rate profiles 202 or 203 may be used along with IOB 220 to easily and safely transition to open-loop operation. Although primarily described herein as pump 12 processor 42 receiving CGM data, calculating therapy parameters, tracking IOB, storing and updating open-loop basal profiles, determining whether therapy should revert to open-loop, etc., in various embodiments a processor of any other device operated as a part of an infusion pump system could provide some or all of these functions. Examples of such devices include for example, a CGM, a smartphone, a dedicated remote controller, an electronic tablet, a computer, etc.

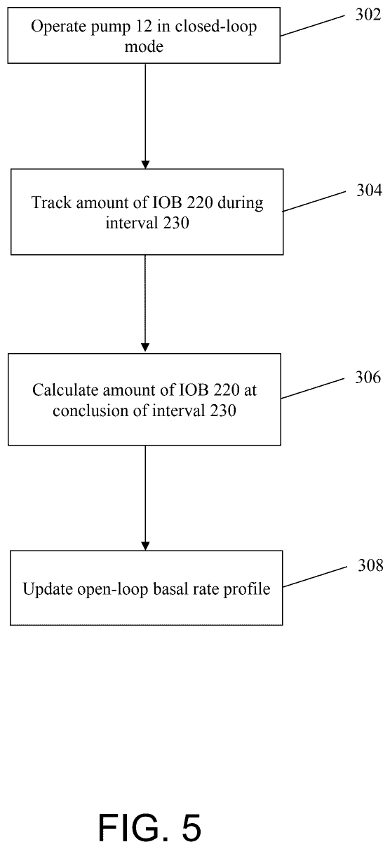

Referring now to FIG. 5, an operational flowchart for pump/processor in closed-loop mode is depicted. At step 302, pump 12 begins operating in closed-loop mode according to an algorithm stored in memory 30 or stored in a separate location and accessible by pump 12. At step 304, the amount of IOB 220 is continually tracked during operation of pump 12 over an interval 230. At step 306, upon the conclusion of interval 230 the amount of IOB 220 is calculated and stored as described herein. At step 308, the open-loop basal rate profile 202 or 203 is updated and stored in memory 30 or stored in a separate location and accessible by pump 12 as described herein.

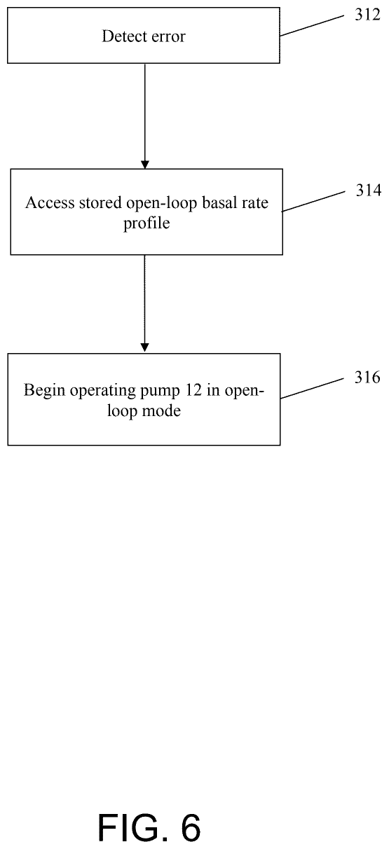

Referring now to FIG. 6, an operational flowchart is depicted upon detection of an error. Such error may include a calibration error with sensor 102 (including, e.g., failure to calibrate the sensor within a time interval, such as a manufacturer's recommended time interval), a failure of sensor 102, an expiration of sensor 102, a loss of signal between sensor 102 and glucose monitoring system 100, a loss of signal between glucose monitoring system 100 and pump 12, or any other errors that may affect the accuracy and safety of delivery of insulin to the patient. Such error may occur at any time during operation of pump 12 in closed-loop mode. At step 314, the open-loop basal rate profile stored in step 308 is accessed. At step 316, pump 12 switches from closed-loop to open-loop operation based at least in part on the amount of IOB calculated in step 306 and/or the open-loop basal rate profile stored in step 308.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 8,287,495; 8,408,421 8,448,824; 8,573,027; 8,650,937; 8,986,523; 9,173,998; 9,180,242; 9,180,243; 9,238,100; 9,242,043; 9,335,910; 9,381,297; 9,421,329; 9,486,171; 9,486,571; 9,492,608; and 9,503,526 commonly owned U.S. Patent Publication Nos. 2009/0287180; 2012/0123230; 2013/0053816; 2013/0324928; 2013/0332874; 2014/0276419; 2014/0276420; 2014/0276423; 2014/0276531; 2014/0276553; 2014/0276556 2014/0276569; 2014/0276570; 2014/0378898; 2015/0073337; 2015/0072613; 2015/0182693; 2015/0182695; 2016/0030669; 2016/0082188; and 2016/0339172 and commonly owned U.S. patents application Ser. Nos. 14/707,851; 15/241,257 and 15/354,495 and commonly owned U.S. Provisional Application Ser. Nos. 61/911,576; 61/920,902; 61/920,914; 61/920,940; 62/139,275; 62/272,255; 62/300,410; 62/352,164; 62/365,167; and 62/394,806.

Further incorporated by reference herein in their entirety are U.S. Pat. Nos. 8,601,465; 8,502,662; 8,452,953; 8,451,230; 8,449,523; 8,444,595; 8,343,092; 8,285,328; 8,126,728; 8,117,481; 8,095,123; 7,999,674; 7,819,843; 7,782,192; 7,109,878; 6,997,920; 6,979,326; 6,936,029; 6,872,200; 6,813,519; 6,641,533; 6,554,798; 6,551,276; 6,295,506; and 5,665,065.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An infusion pump system, comprising:
a pump mechanism configured to facilitate delivery of a medicament to a patient;
a memory adapted to store parameters relating to delivery of the medicament, including an open-loop basal rate profile for the patient;
a communications device adapted to receive information from a continuous glucose monitoring system; and
a processor functionally linked to the pump mechanism, the memory and the communications device to obtain the information from the continuous glucose monitoring system, the processor configured to:
cause the pump mechanism to deliver the medicament to the patient in a closed- loop mode in which therapy parameters are automatically determined and medicament is automatically delivered according to the therapy parameters based on the information from the continuous glucose monitoring system;
continually track an amount of insulin on board in the patient during closed-loop mode;
detect that an error has occurred, the error requiring reversion from closed-loop mode to an open-loop mode; and
cause the pump mechanism to deliver the medicament to the patient in the open- loop mode according to the open-loop basal rate profile stored in memory and the amount of insulin on board in the patient.

2. The infusion pump system of claim 1, wherein the processor is configured to deliver the medicament to the patient in the closed-loop mode for a plurality of time intervals,
wherein continually tracking the amount of insulin on board includes tracking the amount of insulin on board over a first time interval, and
wherein the processor is further configured to modify the open-loop basal rate profile stored in memory after the first time interval based on the tracked amount of insulin on board over the first time interval.

3. The infusion pump system of claim 2, wherein continually tracking the amount of insulin on board includes tracking the amount of insulin on board for each subsequent time interval after the first time interval, and
wherein the processor is configured to modify the open-loop basal rate profile stored in memory after each of the subsequent time intervals based on the tracked amount of insulin on board over each of the respective subsequent time intervals.

4. The infusion pump system of claim 1, wherein the error is selected from the set consisting of: a calibration error of a sensor of the continuous glucose monitoring system, a failure to calibrate the sensor within a calibration time interval, a failure of the sensor, an expiration of the sensor, a loss of signal between the sensor and the continuous glucose monitoring system, a loss of signal between the sensor and the processor, and a loss of signal between the continuous glucose monitoring system and the processor.

5. The infusion pump system of claim 1, wherein the processor is further configured to determine the therapy parameters for the closed-loop mode.

6. The infusion pump system of claim 1, wherein the processor is further configured to receive the therapy parameters for the closed-loop mode from a separate device.

7. The infusion pump system of claim 1, wherein a portion of the continuous glucose monitoring system is incorporated into a common housing with the pump mechanism, memory, communications device and processor, and wherein the communications device is configured to receive the information directly from a sensor of the continuous glucose monitoring system.

8. The infusion pump system of claim 1, wherein the communications device is configured to receive the information from a continuous glucose monitor receiver of the continuous glucose monitoring system.

9. The infusion pump system of claim 1, wherein the processor is further configured to calculate the amount of insulin on board based on a difference between an amount of insulin delivered to the patient over a time interval during closed-loop mode and an amount of insulin in the open-loop basal rate profile over the time interval.

10. An infusion pump system, comprising:
a pump mechanism configured to facilitate delivery of a medicament to a patient;
a memory adapted to store parameters relating to delivery of the medicament, including an open-loop basal rate profile for the patient; and
a processor functionally linked to the pump mechanism and the memory, the processor configured to:
receive information from a continuous glucose monitoring system;
cause the pump mechanism to automatically deliver the medicament to the patient based on therapy parameters automatically determined based on the information from the continuous glucose monitoring system;
continually track an amount of insulin on board in the patient while automatically delivering the medicament based on the therapy parameters;
detect an error pertaining to the continuous glucose monitoring system; and
cause the pump mechanism to cease automatically delivering the medicament based on the therapy parameters and to deliver the medicament according to the open-loop basal rate profile stored in memory and the amount of insulin on board in the patient in response to detection of the error.

11. The infusion pump system of claim 10, wherein the processor is configured to automatically deliver the medicament to the patient based on the therapy parameters for a plurality of time intervals,
wherein continually tracking the amount of insulin on board includes tracking the amount of insulin on board over a first time interval and
wherein the processor is further configured to modify the open-loop basal rate profile stored in memory after the first time interval based on the tracked amount of insulin on board over the first time interval.

12. The infusion pump system of claim 11, wherein continually tracking the amount of insulin on board includes tracking the amount of insulin on board for each subsequent time interval after the first time interval, and
wherein the processor is configured to modify the open-loop basal rate profile stored in memory after each of the subsequent time intervals based on the tracked amount of insulin on board over each of the respective subsequent time intervals.

13. The infusion pump system of claim 10, wherein the error is selected from the set consisting of: a calibration error of a sensor of the continuous glucose monitoring system, a failure to calibrate the sensor within a calibration time interval, a failure of the sensor, an expiration of the sensor, a loss of signal between the sensor and the continuous glucose monitoring system, a loss of signal between the sensor and the processor and a loss of signal between the continuous glucose monitoring system and the processor.

14. The infusion pump system of claim 10, wherein the processor is further configured to determine the therapy parameters.

15. The infusion pump system of claim 10, wherein the processor is further configured to receive the therapy parameters from a separate device.

16. The infusion pump system of claim 10, wherein a portion of the continuous glucose monitoring system is incorporated into a common housing with the pump mechanism, memory and processor, and wherein the processor is configured to receive the information directly from a sensor of the continuous glucose monitoring system.

17. The infusion pump system of claim 10, wherein the processor is configured to receive the information from a continuous glucose monitor receiver of the continuous glucose monitoring system.

18. The infusion pump system of claim 10, wherein the processor is further configured to calculate the amount of insulin on board based on a difference between an amount of insulin delivered to the patient over a time interval when medicament is automatically delivered according to the therapy parameters and an amount of insulin in the open-loop basal rate profile over the time interval.

19. An infusion pump system, comprising:
an infusion pump including a pump mechanism configured to facilitate delivery of a medicament to a patient;
a continuous glucose monitoring system configured to obtain information from which estimated blood glucose levels of the patient are determined; and
a processor configured to:
cause the infusion pump to deliver the medicament to the patient in a closed-loop mode in which therapy parameters are automatically determined and medicament is automatically delivered according to the therapy parameters based on the estimated blood glucose levels;
continually track an amount of insulin on board in the patient during closed-loop mode;
detect that an error has occurred, the error requiring reversion from closed-loop mode to an open-loop mode; and
cause the infusion pump to deliver the medicament to the patient in the open-loop mode according to an open-loop basal rate profile and the amount of insulin on board in the patient.

20. The infusion pump system of claim 19, wherein the processor is disposed in one of the infusion pump, the continuous glucose monitoring system, or a third device separate from the infusion pump and the continuous glucose monitoring system.
